(19) United States
(12) Patent Application Publication
(10) Pub. No.: US 2025/0143701 A1
(43) Pub. Date: May 8, 2025

(54) MOTOR CONTROL OF SURGICAL STAPLER WITH PAUSING IN RESPONSE TO SPEED ERROR

(71) Applicant: CILAG GMBH INTERNATIONAL, ZUG (CH)

(72) Inventors: NICHOLAS J. ROSS, FRANKLIN, OH (US); FREDERICK E. SHELTON, IV, HILLSBORO, OH (US); JASON L. HARRIS, LEBANON, OH (US); KARL W. MUELLER, CINCINNATI, OH (US)

(73) Assignee: CILAG GMBH INTERNATIONAL, ZUG (CH)

(21) Appl. No.: 18/776,686

(22) Filed: Jul. 18, 2024

Related U.S. Application Data
(60) Provisional application No. 63/587,193, filed on Oct. 2, 2023.

(51) Int. Cl. A61B 17/072; A61B 17/00; G16H 20/30; G16H 40/63

(52) U.S. Cl. CPC A61B 17/07207; G16H 20/30; G16H 40/63; A61B 2017/0019; A61B 2017/00398

(57) ABSTRACT

A method of driving a motor in a powered surgical stapler can include a pausing monitoring process that may be effective to improve staple form and/or increase localized compression of tissue. The pausing monitoring process monitors firing speed of a firing bar driving by the motor and pauses the motor when the firing speed passes a speed error threshold. Before pausing the motor, the pausing monitoring process may also require that a pulse width modulated (PWM) electrical signal driving the motor has a duty cycle over a duty cycle threshold while the firing speed is beyond the speed error. The pausing monitoring process may force the firing bar through a predetermined distance immediately after pausing. The pausing monitoring process may be limited in the number of pauses that can be taken during a firing stroke.

### CROSS REFERENCE TO RELATED APPLICATIONS

This application claims benefit of priority to prior filed U.S. Provisional Patent Application No. 63/587,193 filed Oct. 2, 2023, which is hereby incorporated by reference in full herein.

### FIELD

This application relates generally to medical devices, and in particular to motor driven surgical staplers.

### BACKGROUND

Innovation in surgical stapling technology has evolved from manual to power-operated staplers. Manual staplers clamp tissue, deliver staples, and drive a knife blade through mechanical force applied to lever(s) on a handle of the stapler. Powered staplers use an electrically powered motor to drive the knife blade and staples. Powered staplers may also use an electrically powered motor to clamp tissue. Upon their introduction, powered staplers delivered staples at fixed speed without regard to tissue properties. Many surgical stapling challenges relate to tissue dynamics, tissue movement, and tissue variability. Different tissue types present unique tissue challenges. Firing algorithms have been disclosed in which motor drive is varied with time to address the unique tissue challenges. For instance, U.S. Pat. No. 10,307,170 discloses a method for closed loop control of motor velocity of a surgical stapler; U.S. Pat. No. 10,368,865 discloses mechanisms for compensating for drivetrain failure in powered surgical instruments; U.S. Pat. No. 11,090,046 discloses systems and methods for controlling displacement member motion of a surgical stapler; U.S. Patent Pub. No. 2023/0048444 discloses a variable response motor control algorithm for a powered surgical stapler; U.S. Pat. No. 10,828,028 discloses a surgical instrument with multiple program responses during a firing motion; U.S. Pat. No. 9,808,246 discloses a method of operating a powered surgical instrument; and U.S. Pat. No. 9,016,540 discloses a device and method for controlling compression of tissue, each of which are incorporated by reference as if set forth in their entireties herein.

### SUMMARY

Examples disclosed herein generally describe software for driving a motor in a powered surgical stapler to provide consistent stapling performance, specifically on thick tissue. The software includes a pausing monitoring process that may be effective to improve staple form and/or increase localized compression of tissue. The pausing monitoring process monitors firing speed of a firing bar driven by the motor and pauses the motor when the firing speed passes a speed error threshold. Before pausing the motor, the pausing monitoring process may also require that a pulse width modulated (PWM) electrical signal driving the motor has a duty cycle over a duty cycle threshold while the firing speed is beyond the speed error. The pausing monitoring process may force the firing bar through a predetermined distance immediately after pausing. The pausing monitoring process may be limited in the number of pauses that can be taken during a firing stroke.

In one embodiment, a surgical stapler includes a firing assembly, a motor assembly, and a speed control circuit. The firing assembly is configured to translate along a longitudinal axis such that translation of the firing assembly in a distal direction is configured to deploy staples from an end effector. The motor assembly is mechanically coupled to the firing assembly and configured to drive the firing assembly along the longitudinal axis. The speed control circuit is configured to output a motor drive signal to the motor assembly such that the motor drive signal is configured to drive the firing assembly to a target speed during a firing stroke, detect, during the firing stroke, a speed error in which an actual speed of the firing assembly is less than a speed threshold that is less than the target speed, set the target speed to zero for a pause time duration in response to detecting the speed error, increase the target speed above zero after the pause time duration, and drive the firing assembly to the increased target speed a predetermined distance through the firing stroke.

In one embodiment, a method for controlling speed of a firing stroke of a surgical stapler includes: outputting a motor drive signal to a motor assembly such that the motor drive signal is configured to drive a firing assembly of the surgical stapler to a target speed during the firing stroke; detecting, during the firing stroke, a speed error in which an actual speed of the firing assembly is less than a speed threshold that is less than the target speed; setting the target speed to zero for a pause time duration in response to detecting the speed error; increasing the target speed above zero after the pause time duration; and driving the firing assembly to the increased target speed a predetermined distance through the firing stroke.

In one embodiment, a method for controlling speed of a firing stroke of a surgical stapler includes: outputting a motor drive signal to a motor assembly such that the motor drive signal is configured to drive a firing assembly of the surgical stapler to a target speed during the firing stroke; detecting, during the firing stroke, a speed error in which an actual speed of the firing assembly is less than a speed threshold that is less than the target speed and a parameter of the motor drive signal is beyond a threshold value; setting the target speed to zero for a pause time duration in response to detecting the speed error; increasing the target speed above zero after the pause time duration; and driving the firing assembly to the increased target speed a predetermined distance through the firing stroke.

### BRIEF DESCRIPTION OF THE DRAWINGS

While the specification concludes with claims, which particularly point out and distinctly claim the subject matter described herein, it is believed the subject matter will be better understood from the following description of certain examples taken in conjunction with the accompanying drawings, in which like reference numerals identify the same elements. The figures depict one or more implementations of the inventive devices, by way of example only, not by way of limitation.

FIG. 1 is a perspective view of an exemplary powered surgical stapler.

FIG. 2 is an illustration of an exemplary end effector of an exemplary powered surgical stapler.

FIG. 3 is a block diagram of an exemplary firing driver for a powered surgical stapler.

FIG. 4 is an illustration of a flow diagram of an exemplary software algorithm for driving a motor of a powered surgical stapler.

FIG. 5A is a graph of firing speed as a function of firing assembly position.

FIG. 5B is a graph of motor power as a function of firing assembly position.

FIG. 5C is a graph of firing force as a function of firing assembly position.

FIG. 6 is a graph of firing speed as a function of firing assembly position, illustrating speed control variables.

FIG. 7 is a flow diagram of a method for controlling speed of a firing stroke of a surgical stapler.

FIG. 8 is another exemplary flow diagram of a method for controlling speed of a firing stroke of a surgical stapler.

FIG. 9A is an exemplary flow diagram illustrating the first control loop in FIG. 8.

FIG. 9B is a graph of an initiated pause and an increase of power threshold during a portion of a firing stroke.

FIG. 10A is an exemplary flow diagram illustrating the second control loop in FIG. 8.

FIG. 10B is a graph of an initiated pause and a change of target speed during a portion of a firing stroke.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives, and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±10% of the recited value, e.g., “about 90%” may refer to the range of values from 81% to 99%.

As used herein, the terms “patient,” “host,” “user,” and “subject” refer to any human or animal subject and are not intended to limit the systems or methods to human use, although use of the subject invention in a human patient represents a preferred embodiment. As well, the term “proximal” indicates a location closer to the operator whereas “distal” indicates a location further away to the operator or physician.

As used herein, the term “memory” and “non-transitory computer-readable media” are used interchangeable and are understood to include, but are not limited to, random access memory (RAM), read-only memory (ROM), electronically erasable programmable ROM (EEPROM), flash memory or other memory technology, compact disc ROM (CD-ROM), digital versatile disks (DVD) or other optical storage, magnetic cassettes, magnetic tape, magnetic disk storage or other magnetic storage devices, or any other tangible, physical medium which can be used to store computer readable information.

Alternative apparatus and system features and alternative method steps are presented in example embodiments herein. Each given example embodiment presented herein can be modified to include a feature and/or method step presented with a different example embodiment herein where such feature and/or step is compatible with the given example as understood by a person skilled in the pertinent art as well as where explicitly stated herein. Such modifications and variations are intended to be included within the scope of the claims.

Examples disclosed herein generally describe software for driving a motor in a powered surgical stapler to provide consistent stapling performance, specifically on thick tissue. A stapler configured with the described software may provide reliable staple formation, deliver consistent results, support better patient outcomes, and/or provide other benefits. The software preferably can be utilized with existing powered surgical stapler hardware such as the ECHELON™ 3000 and other contemporary powered surgical staplers. Additionally, or alternatively, the software may be compatible with older powered surgical staplers, robotic surgical staplers, and surgical stapler hardware yet to be developed. The software may have access to multiple inputs (time, displacement, speed, force, power, etc.), and may control motor speed based on some or all of the inputs. The software includes a pausing monitoring process that may be effective to improve staple form. Introducing pausing allows the system to relax and increase localized compression of tissue during a staple firing stroke.

In some embodiments, the motor of the powered surgical stapler is configured to translate a firing bar longitudinally. In some embodiments, the software is configured to drive the motor so that the firing bar travels at a fixed initial speed of 12 mm/s. Alternatively, the initial speed may be as fast as 16 mm/s; however 12 mm/s was determined better than 16 mm/s in speed design of experiments (DoE) testing to show more consistent staple form results using the ECHELON™ 3000 powered surgical stapler.

In some embodiments, the motor of the powered surgical stapler is driven by a pulse width modulated (PWM) electrical signal in which the duty cycle of the PWM signal can be adjusted to vary power delivery to the motor. In some embodiments, the maximum speed of the firing bar is approximately 12 mm/s because the duty cycle of the PWM electrical signal reaches 100% PWM at a firing bar speed of approximately 12 mm/s in an unloaded firing condition.

Alternatively, the firing bar speed may be significantly greater than 12 mm/s when the motor is driven by a PWM electrical signal having 100% duty cycle. In this embodiment, the software can be configured with a duty cycle threshold.

In some embodiments, the software may include a speed error threshold that may be determined as a static parameter or assigned to powered surgical stapler during an instrument-specific characterization process. The software can monitor the speed of the firing bar and pause the firing bar when the speed passes the speed error threshold. The software reduces the speed of the firing bar to zero during the pause. During the pause, tissue is compressed within opposite components (e.g., jaws) of the end effector of the powered surgical stapler. Preferably, the duration of each pause is approximately one second.

In some embodiments, the software algorithm can force the firing bar through a predetermined distance (“disabled zone”) immediately following a pause. The software can be prevented from pausing due to speed error until after the firing bar travels the predetermined distance. This forces the powered surgical stapler to proceed with an uninterrupted knife firing until the firing bar travels the predetermined distance of the “disabled zone”. This feature of the algorithm can be effective to maximize tissue compression due to pausing while preventing diminishing returns from excess pause actuation.

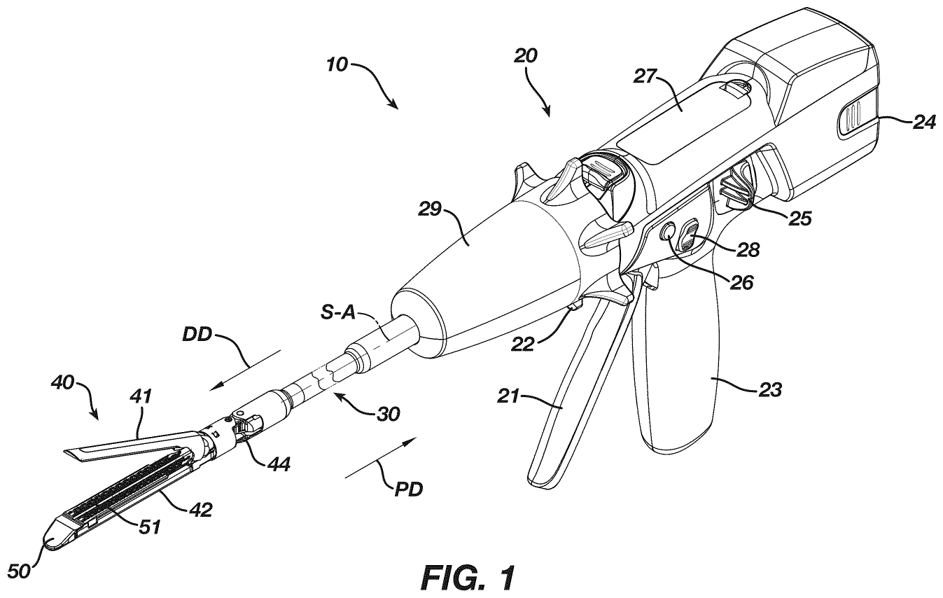

FIG. 1 is a perspective view of an exemplary surgical stapler 10 including a handle 20, a shaft 30, and an end effector 40. The handle 20 is configured to be grasped, manipulated, and actuated by a clinician. The shaft 30 is sized, shaped, and otherwise configured to extend through a body opening of the patient. The end effector 40 is configured deliver staples 51. The end effector 40 may also be configured to cut tissue within the body of the patient.

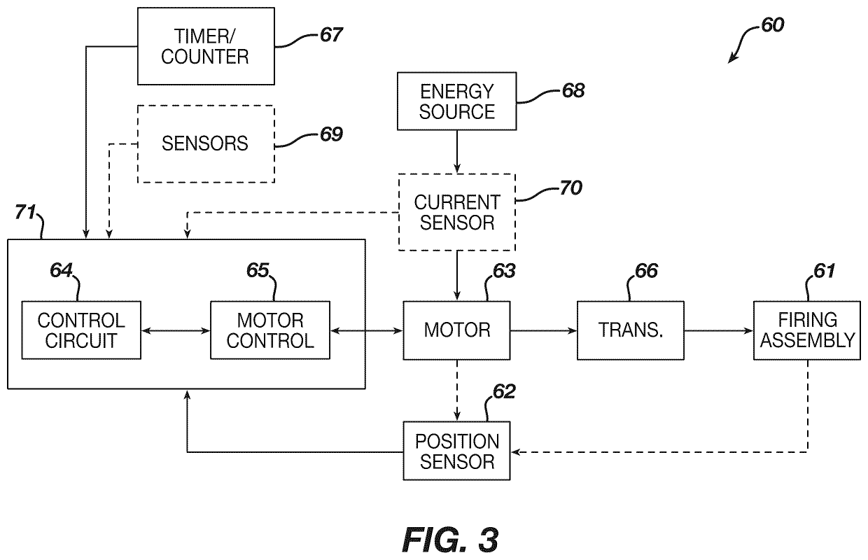

The handle 20 can include a closure trigger 21, a firing trigger 22, and a grip 23 sized such that a clinician can single-handedly hold the surgical stapler 10 by the grip 23 while manipulating the closure trigger 21 or the firing trigger 22. The closure trigger 21 is operably connected to a motor disposed within the handle 20 such that when the closure trigger 21 is pulled, the motor is driven to cause the end effector 40 to clamp tissue. Alternatively, the closure trigger 21 can be mechanically coupled to the end effector 40 such that when the closure trigger 21 is pulled, the end effector 40 moves to clamp tissue without the aid of a motor. The firing trigger 22 is operably connected to a motor 63 (FIG. 3) disposed within the handle 20 such that when the firing trigger 22 is pulled, the motor 63 is driven to cause the end effector 40 to deploy staples 51 into the clamped tissue and may also cut the clamped tissue.

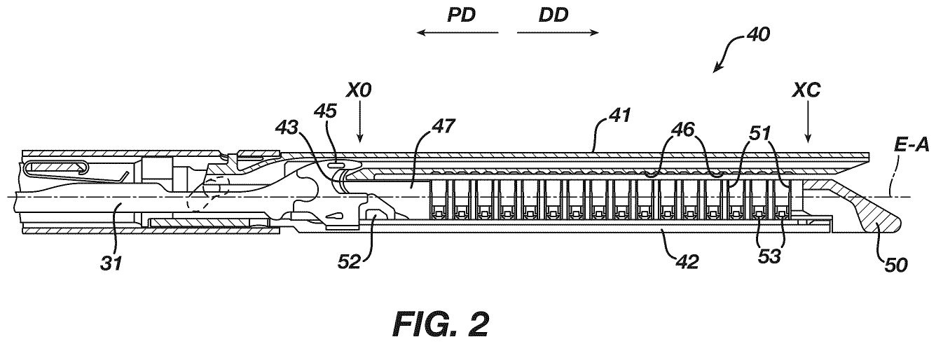

The handle 20 can further include additional features such as a firing trigger lock mechanism (not illustrated) which can be manipulated to prevent actuation of the firing trigger 22, a power pack 24 configured to provide electrical power to the motor and other electrical components of the powered surgical stapler 10, a closure release button 25 which can be manipulated to release the end effector 40 and the closure trigger 21 from the clamped position, a home button 26 that can be pressed to cause the motor to move a knife 43 (FIG. 2) of the end effector 40 in the proximal direction PD to a home position (FIG. 2), a manual override 27 including a mechanical actuator which can be manipulated to mechanically move the knife proximally to the home position, articulation buttons 28 that can be pressed to cause a motor to articulate the end effector 40 at an articulation joint 44 so that the end effector 40 is at an angle with a longitudinal axis S-A of the shaft 30, a rotatable nozzle 29 configured to be rotated so that the shaft 30 and end effector rotate about the shaft axis S-A, a display (not illustrated) configured to display information related to the surgical stapler, variations thereof, other compatible features of a powered surgical stapler handle, and combinations thereof.

The end effector 40 includes an anvil 41 and a staple jaw 42 opposite the anvil 41. The anvil 41 and staple jaw 42 are illustrated in an open position in FIG. 1. The anvil 41 and staple jaw 42 can be moved toward each other to move the end effector 40 to a clamped configuration. For instance, tissue (not illustrated) can be positioned between the anvil 41 and staple jaw 42 in the open position, and the anvil 41 can rotate toward the staple jaw 42 to clamp the tissue.

When the end effector 40 is in the clamped configuration (FIG. 2), the firing trigger 22 can be pulled to cause deployment of staples 51 (FIG. 2) from the cartridge 50 and may also cause cutting of tissue by driving knife 43.

Portions of the surgical stapler 10 may be detachable and interchangeable. Staples 51 may be housed in a staple cartridge 50 that is detachable from the end effector 40. The end effector 40 may be detachable from the shaft 30, and the shaft 30-handle 20 combination may be configured for use in connection with interchangeable end effectors. At least a portion of the shaft 30 including the end effector 40 may be detachable from the handle 20, and the handle 20 may be configured for use in connection with interchangeable shaft assemblies having different shaft lengths and/or different end effectors attached thereto.

FIG. 2 is a sectional view of the end effector 40 of the powered surgical stapler 10. The end effector 40 is in a clamped configuration and the knife 43 is at the home position X0 prior to a firing stroke. The staple cartridge 50 is attached to the staple jaw 42 and positioned within a cartridge channel 47. A firing bar 31 extends in the proximal direction PD into the shaft 30. The firing bar 31 is translatable in the distal direction DD and the proximal direction PD. A distal portion of the firing bar 31 moves through the end effector 40 along a longitudinal axis E-A of the end effector 40. When the end effector 40 is not articulated, the longitudinal axis E-A of the end effector 40 is in line with the shaft axis S-A (FIG. 1). An I-beam 45 is coupled to the knife 43 and the distal portion of the firing bar 31. A wedge sled 52 is positioned in the staple cartridge 50. As the I-beam 45 translates distally, the cutting edge of the knife 43 contacts and may cut tissue positioned between the anvil 41 and the staple cartridge 50. Also, the I-beam 45 contacts the wedge sled 52 and pushes it distally, causing the wedge sled 52 to contact staple drivers 53. The staple drivers 53 may be driven up into staples 51, causing the staples 51 to advance through tissue and into pockets 46 defined in the anvil 41, which shape the staples 51.

The knife 43 and I-beam 45 are at the home position X0 at before advancing through a firing stroke. During a firing stroke, the I-beam 45 and knife 43 move distally from the home position X0. The firing stroke is completed when the I-beam 45 and knife 43 arrive at the stroke end position, or complete position XC. The length of the firing stroke is therefore the distance from the home position X0 to the complete position XC. When the I-beam 45 and knife 43 are at the complete position, all staples 51 of the staple cartridge 50 have been deployed.

During a firing stroke, several components of the powered surgical stapler 10 translate longitudinally (i.e., along the shaft axis S-A and/or along the end effector axis E-A), including the firing bar 31, the knife 43, the I-beam 45, and the wedge sled 52. The components of the powered surgical stapler 10 which translate longitudinally during a firing stroke are collectively referred to herein as a “firing assembly”. The end effector 40 illustrated in FIG. 2 can be modified to include additional and/or alternative firing assembly components as understood by person skilled in the pertinent art. Further, the shaft 30 and/or handle 20 may include additional firing assembly components not illustrated herein as understood by a person skilled in the pertinent art.

The speed at which at least a portion of the components of the firing assembly translate longitudinally during a firing stroke is related to the firing speed in a deterministic way. As illustrated, the speed at which the distal portion of the firing bar 31, the knife 43, the I-beam 45, and the wedge sled 52 translate longitudinally is equal to the firing speed. Optionally, the firing assembly may include components such as springs or gears which cause at least a portion of the components of the firing assembly to translate longitudinally at a speed that is not equal to the firing speed.

The firing assembly may also include a component configured to maintain the end effector 40 in a clamped configuration during a firing stroke. As illustrated, the I-beam 45 is configured to translate through respective channels in the anvil 41 and staple jaw 42 during a firing stroke to maintain the end effector 40 in the clamped configuration during the firing stroke. The firing assembly can be modified to include additional or alternative components configured to maintain the end effector 40 in a clamped configuration during a firing stroke as understood by a person skilled in the pertinent art. In an alternative embodiment, the firing assembly need not include a component configured to maintain the end effector 40 in a clamped configuration; and the powered surgical stapler 10 can be modified to include a clamping component that does not translate longitudinally during a firing stroke. In such an embodiment, the knife 43 may also be omitted.

FIG. 3 is block diagram of an example firing driver 60 for a powered surgical stapler such as the powered surgical stapler 10 illustrated in FIG. 1, variations thereof, or an alternative thereto as understood by a person skilled in the pertinent art. The firing driver 60 is configured to control longitudinal translation of a firing assembly 61 of the powered surgical stapler 10. The firing assembly 61 may include the firing bar 31, the knife 43, the I-beam 45, and the wedge sled 52 illustrated in FIG. 2, alternatives thereto, variations thereof, and/or sub combinations thereof as understood by a person skilled in the pertinent art. The firing assembly 61 is configured to deploy staples and may also cut tissue during a firing stroke of the powered surgical stapler 10. The firing driver 60 includes a speed control circuit 71 configured to drive the motor 63. The firing driver 60 includes a transmission 66 configured to convert the rotational movement of a rotor of the motor 63 into longitudinal movement of the firing assembly 61. The motor 63 and transmission 66 are collectively referred to herein as a motor assembly. Note that the motor 63 as illustrated, may represent more than one motor.

The position, movement, displacement, and/or translation of one or more components of the firing assembly 61, can be measured by one or more position sensors 62. The position sensor(s) 62 may be configured to detect movement of the firing assembly 61 and/or rotation of the rotor of the motor 63. The position sensor(s) 62 can otherwise be configured to sense a physical parameter of the powered surgical stapler 10 and provide an electrical signal output indicative of the knife 43, I-beam 45, wedge sled 52, or other portion of the firing assembly 61 which translates longitudinally through the end effector 40 during a firing stroke. Additionally, or alternatively, the position sensor 62 can be configured to detect which staples 51 have been deployed and which have not been deployed. Deployment status of staples 51 may provide an indication of a position of the distal portion of the firing assembly 61.

The position sensor(s) 62 may be located in the end effector 40 and/or at any other portion of the powered surgical stapler 10. In some embodiments, the position sensor(s) 62 include an encoder configured to provide a series of pulses to the speed control circuit 71 as the rotor of the motor 63 rotates and the firing assembly 61 is translated longitudinally. The speed control circuit 71 may track the pulses to determine the position of a component of the firing assembly 61 (e.g., firing bar 31, knife 43, I-beam 45, and/or wedge sled 52). Other suitable position sensors may be used, including, for example, a proximity sensor. Other types of position sensors may provide other signals indicating motion of a component of the firing assembly 61. In some embodiments, the position sensor(s) 62 may be omitted. For instance, where the motor 63 is a stepper motor, the speed control circuit 71 may track the position of a component of the firing assembly 61 by aggregating the number and direction of steps that the motor 63 has been instructed to execute.

The speed control circuit 71 is illustrated as including a control circuit 64 and motor controller 65, which are illustrated as two separate blocks. The control circuit 64 and motor controller 65 and may be separate circuits or may be integrated as a single circuit. The control circuit 64 is configured to provide a motor setpoint signal output to the motor controller 65. The motor setpoint signal is indicative of a target speed of the firing assembly 61. The motor controller 65 is configured to provide a motor drive signal to the motor 63 such that the motor drive signal is based on the motor setpoint signal and intended to drive the motor 63 so that the firing assembly 61 is driven to the target speed.

Note that, during a firing stroke, the actual speed of the firing assembly 61 may not precisely match the target speed. The motor controller 65 is configured to drive the firing assembly 61 to the target speed, meaning, as the actual speed of the firing assembly 61 deviates from the target speed, the motor controller 65 is configured to adjust the speed of the firing assembly 61 so that the speed of the firing assembly more closely matches the target speed.

The control circuit 64 and the motor controller 65 may include one or more processors and memory (i.e., one or more non-transitory computer-readable medium) with instructions that can be executed by the one or more processors to cause the control circuit 64 and the motor controller 65 to drive the motor 63. The control circuit 64 and/or motor controller 65 can include a feedback controller, which can be one of any feedback controllers, including, but not limited to a PID, a State Feedback, LQR, and/or an Adaptive controller, for example. The control circuit 64 and/or motor controller 65 can include a power source to convert the signal from the feedback controller into a physical input such as a constant voltage, pulse width modulated (PWM) voltage, frequency modulated voltage, current, torque, and/or force, for example.

The firing driver 60 includes a timer/counter circuit 67 configured to provide an output signal, such as elapsed time or a digital count, to the control circuit 64. The control circuit 64 is configured to determine a position of the firing assembly 61 based on the signal from the position sensor(s) 62 and correlate the position of the firing assembly 61 with the output of the timer/counter circuit 67 such that the control circuit 64 can determine the position of one or more components of the firing assembly 61 (e.g. firing bar 31, knife 43, I-beam 45, and/or wedge sled 52) at a specific time relative to a starting position X0 (FIG. 2). The timer/counter circuit 67 may be configured to measure elapsed time, count external events, or time external events.

At the beginning of a firing stroke the control circuit 64 can be configured to provide a motor set point signal to the motor control 65 that indicates a fixed initial speed. The motor controller 65 can be configured to provide a motor drive input signal to the motor 63 that adjusts power drawn by the motor 63 so that the motor 63 is driven approximately at the fixed initial speed. In some embodiments, the fixed initial speed is approximately 12 mm/s to approximately 16 mm/s, and more preferably at approximately 12 mm/s.

In some embodiments, the speed control circuit 71 is configured to set the target speed to the initial speed such that the firing assembly 61 traverses an initial distance of the firing stroke, driven to the initial speed.

In some embodiments, the motor 63 is driven by a pulse width modulated (PWM) electrical signal in which the duty cycle of the PWM signal can be adjusted by the motor controller 65 to vary power delivered to the motor 63. The motor controller 65 may include one or more electrical circuits configured to provide a motor drive signal to the motor 63. In some embodiments, the motor 63 can include a brushless direct current (DC) electric motor and the motor control 65 may provide a PWM motor drive signal to one or more stator windings of the motor 63.

In some embodiments, the firing driver 60 is configured such that the PWM electrical signal is at 100% duty cycle when the firing assembly 61 is driven at the fixed initial speed and the firing assembly 61 in uninhibited by external factors such as tissue thickness. In this embodiment, the motor 63 is driven by a 100% duty cycle PWM signal (i.e., direct current (DC) signal) at the beginning of the firing stroke. Alternatively, the firing driver 60 may be configured such that the PWM electrical signal is at less than 100% duty cycle when the firing assembly 61 is driven at the fixed initial speed and the firing assembly 61 in uninhibited by external factors such as tissue thickness. In this embodiment, the motor 63 is driven by a PWM signal having a duty cycle of less than 100% at the beginning of the firing stroke. Further, the motor controller 65 may be configured with a duty cycle threshold.

In some embodiments, the motor controller 65 is configured to provide the PWM signal output to the motor 63 that has a fixed duty cycle corresponding to a target speed provided by the motor set point signal from the control circuit 64.

In some embodiments, the motor controller 65 may provide a variable duty cycle PWM signal output to the motor 63 that is adjusted based on speed error. For instance, the motor controller 65 can be configured to compare an actual speed of the firing assembly to the target speed provided by the motor set point signal from the control circuit 64 and vary the duty cycle of the motor drive signal in response to error between the target speed and actual speed. The actual speed can be provided from the control circuit 64 and/or may be determined based on measurements from the position sensor(s) 62 and timer/counter 67 as disclosed herein and otherwise understood by a person skilled in the pertinent art.

In some embodiments, the motor controller 65 includes a closed loop feedback system that adjusts or controls the duty cycle of the motor drive signal to adjust the speed of the firing assembly 61 based on a magnitude of one or more feedback error terms over a specified increment of either time or distance. The feedback error terms of interest may include, for example, short term, rate of change, steady state, and accumulated. Different feedback error terms can be used in different zones (e.g., during acceleration, initial stabilization, and steady state). Different feedback error terms can be magnified differently based on their importance within the algorithm. Examples of feedback error terms are illustrated in FIG. 6.

In some embodiments, the control circuit 64 is configured to detect a speed error when an actual speed of the firing assembly is less than a speed threshold that is less than the target speed. The speed threshold may be determined as a static parameter or assigned to powered surgical stapler 10 during an instrument-specific characterization process. The control circuit 64 can be configured to determine the speed of the firing assembly 61 based on signal from the timer/counter 67 and the position sensor 62 and provide an output signal to the motor control 65 to pause the firing assembly 61 when the speed passes the speed threshold.

In some embodiments, the motor drive signal provided from the motor controller 65 to the motor 63 includes a PWM signal. The control circuit 64 can be configured to detect that the duty cycle of the PWM signal is greater than a duty cycle threshold. In some embodiments, the control circuit 64 is configured to detect the speed error based on the speed threshold and the duty cycle threshold. The duty cycle threshold may be determined as a static parameter or assigned to a power surgical stapler 10 during an instrument-specific characterization process. The motor controller 65 can be configured to provide an electrical signal to the control circuit 64 indicative of the duty cycle of the PWM signal driving the motor 63. The control circuit 64 can be configured to simultaneously monitor the speed of the firing assembly 61 and the duty cycle of the motor drive signal.

In some embodiments, the control circuit 64 can be configured to initiate a pause in response to detecting the speed error. The control circuit 64 is configured to set the target speed to zero for a pause time duration in response to detecting the speed error. The motor setpoint signal output from the control circuit 64 to the motor controller 65 indicates a target speed of zero during the pause time duration. During a pause, tissue is compressed between the anvil 41 and staple cartridge 50 of the end effector 40 of the powered surgical stapler 10. During at least a portion of the pause, the firing assembly 61 remains stationary at a paused position. During the pause, a distal portion of the firing assembly 61 (e.g., knife 43 and I-beam 45) is positioned between the home position X0 and complete position XC (FIG. 2). The firing assembly may decelerate during an initial time duration of the pause. The pause preferably has a duration of about 1 second.

In some embodiments, the control circuit 64 is configured to increase the target speed above zero after the pause time duration. The control circuit 64 can be configured to provide a motor set-point signal indicating a non-zero speed immediately following a pause.

In some embodiments, the speed control circuit 71 is configured to drive the firing assembly to the increased target speed a predetermined distance through the firing stroke. The control circuit 64 can be configured to monitor change in position of the firing assembly 61 based on signals from the position sensor(s) 62 and retain the increased target speed at least until the firing assembly 61 has travelled distally through the predetermined distance (“disabled zone”) from the paused position. The motor controller 65 can be configured to drive the firing assembly 61 to the increased target speed as indicated by the control circuit 64. Although speed error may occur as the firing assembly 61 travels through the disabled zone, the control circuit 64 is not configured to pause in response to the speed error while the firing assembly 61 is in the disabled zone. This forces the powered surgical stapler 10 to proceed with an uninterrupted staple/knife firing until the firing assembly 61 travels the predetermined distance of the “disabled zone”.

In some embodiments, the increased (non-zero) target speed immediately following the pause may be less than the initial target speed at the beginning of the firing stroke. The control circuit 64 can be configured to provide a motor set-point signal following a pause such that the motor set-point signal indicates a speed that is less than a speed indicated by the motor set-point signal prior to the pause.

In some embodiments, the control circuit 64 is configured with a pause count threshold. The control circuit 64 is configured to complete the firing stroke without initiating a pause when the number of pauses taken during the firing stroke is at the pause count threshold. The control circuit 64 of the speed control circuit 71 can be configured to count a number of pause time durations that occur during a given firing stroke, and the motor controller 65 of the speed control circuit 71 can be configured to drive the firing assembly to the increased target speed through completion of the firing stroke when the number of pause time durations is above the pause count threshold.

The control circuit 64 may optionally be in communication with one or more sensors 69. The sensors 69 may be positioned on the end effector 40 and configured to measure the various derived parameters such as gap distance (between anvil and cartridge) versus time, tissue compression versus time, and anvil strain versus time. The sensors 69 may include a magnetic sensor, a magnetic field sensor, a strain gauge, a pressure sensor, a force sensor, an inductive sensor such as an eddy current sensor, a resistive sensor, a capacitive sensor, an optical sensor, and/or any other suitable sensor for measuring one or more parameters of the end effector 40.

The firing driver 60 may optionally include a current sensor 70 configured to measure the current drawn by the motor 63 from the energy source 68. In some embodiments the control circuit 64 is configured to detect the speed error when the actual speed of the firing assembly 61 is less than the target speed by the speed threshold and electrical current driving the motor 63 is greater than a current threshold.

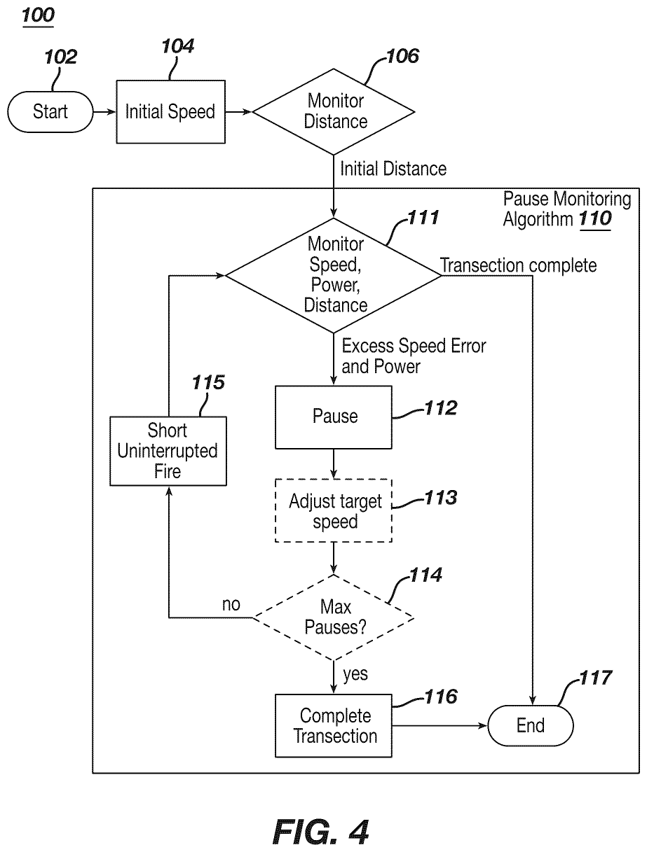

FIG. 4 is an illustration of a flow diagram of an exemplary software algorithm 100 for driving a motor of a powered surgical stapler. The software algorithm 100 is suitable for the powered surgical stapler 10 illustrated herein, the ECHELON™ 3000 powered surgical stapler, variations thereof and alternatives thereto as understood by a person skilled in the pertinent art. The software algorithm 100 can be executed by the firing driver 60 illustrated herein, variations thereof and alternatives thereto as understood by a person skilled in the pertinent art. For instance, the controller circuit 64 (FIG. 3) can include one or more processors and memory with instructions thereon, that when executed by the processor, cause the controller circuit 64 to update the motor setpoint signal output according to the software algorithm 100. This, in turn can initiate pauses and adjust the speed of a firing stroke.

At the start block 102, the powered surgical stapler 10 (FIG. 1) is ready to proceed with a firing stroke. A knife 43, I-beam 45, or other distal portion of a firing assembly 61 may be at a home position X0 in the end effector 40 (FIGS. 2 and 3).

At block 104, the software algorithm 100 provides an initial target firing speed. In some embodiments, the initial target firing speed is approximately 12 mm/s to approximately 16 mm/s, and more preferably at approximately 12 mm/s.

At block 106, the software algorithm 100 maintains the initial target firing speed while monitoring distance of travel of the firing assembly 61. The distance of travel can be determined by the control circuit 64 based on a signal provided by the position sensor 62 (FIG. 3), otherwise monitored as disclosed herein, and/or otherwise monitored as understood by a person skilled in the pertinent art. The firing assembly 61 travels from a home position through an initial distance. The software algorithm 100 determines the firing assembly is at or beyond the initial distance and proceeds to block 111 of a pause monitoring algorithm 110.

At block 111, the pause monitoring algorithm 110 monitors speed, power, and distance. The pause monitoring algorithm 110 can be configured with a speed error threshold that may be determined as a static parameter or assigned to the powered surgical stapler 10 during an instrument-specific characterization process. The pause monitoring algorithm 110 can be configured with a power threshold that may be determined as a static parameter or assigned to the powered surgical stapler 10 during an instrument-specific characterization process. Power to a motor driving the firing assembly (e.g., motor 63 driving firing assembly 61) and the power threshold can be based on duty cycle of a PWM signal driving the motor, current draw by the motor, or other suitable parameter as understood by a person skilled in the pertinent art. The monitored distance can be a distance traveled by some or all of the components of the firing assembly 61 as measured from the beginning of the firing stroke, as measured from the initial distance, as measured from a paused location, as measured from another intermediate point in the firing stroke, and/or as measured from a position at the completion of the firing stroke.

If the pause monitoring algorithm 110 determines that a condition with excess speed error and (optionally) excess power is present, the pause monitoring algorithm proceeds to block 112. Otherwise, if the pause monitoring algorithm 110 determines that the monitored distance is commensurate with the completion of a firing stroke or otherwise determines that transection is complete (e.g., due to an intervention during a firing stroke such as a bailout error), the pause monitoring algorithm 110 ends at end block 117.

At block 112, the pause monitoring algorithm enters a paused state. During the paused state, the target speed for the firing assembly 61 is zero. During at least a portion of the paused state, the firing assembly 61 does not travel any additional distance and is stationary at a paused location.

At block 113, optionally, the pause monitoring algorithm 110 may update the target firing speed. The updated target firing speed may be less than the initial target firing speed. The updated target firing speed may be less than the target firing speed of the algorithm 100 prior to the pause at block 112.

At block 114, the pause monitoring algorithm 110 determines whether the number of pauses taken during the firing stroke has reached the pause count threshold. If the pause count threshold has been reached, the pause monitoring algorithm 110 proceeds to block 116, if not, then the pause monitoring algorithm 110 proceeds to block 115.

At block 115, the pause monitoring algorithm 110 initiates a short uninterrupted firing stroke portion in which the firing assembly travels a predetermined distance without the pause monitoring algorithm 110 initiating a pause. The target firing speed is non-zero during the short uninterrupted firing stroke portion. The distance of travel of the firing assembly 61, from the most recent paused position of the firing assembly 61, is monitored by the pause monitoring algorithm 110. The predetermined distance can be set based on the distance through the firing stroke. For instance, the predetermined distance may be set where the firing assembly 61 does not pass the endpoint of the firing stroke. When the distance from the most recent paused position reaches the predetermined distance, the pause monitoring algorithm 110 proceeds to block 111 described above.

The pause monitoring algorithm 110 iteratively repeats the pause loop including blocks 111, 112, 113, 114, and 115 until either (1) the pause count threshold has been reached at block 114; or (2) transection is complete. When the pause count threshold has been reached at block 114, the pause monitoring algorithm 110 proceeds to block 116.

At block 116, the firing stroke is continued without entering the pause loop. If the pause monitoring algorithm 110 determines that the monitored distance is commensurate with the completion of a firing stroke or otherwise determines that transection is complete (e.g., bailout error), the pause monitoring algorithm 110 ends at end block 117.

At end block 117, the powered surgical stapler 10 may engage another algorithm to execute functions of the powered surgical stapler such as retraction of the firing assembly 61.

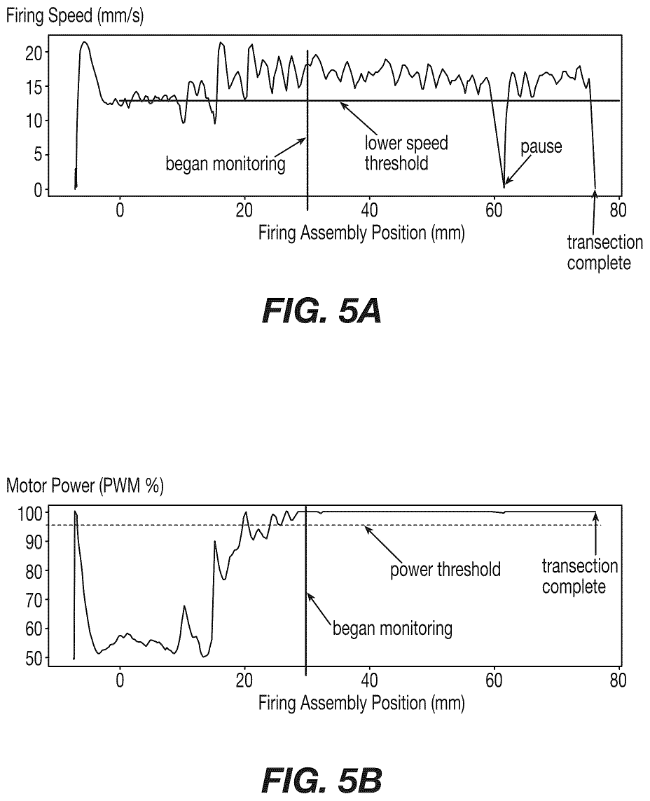

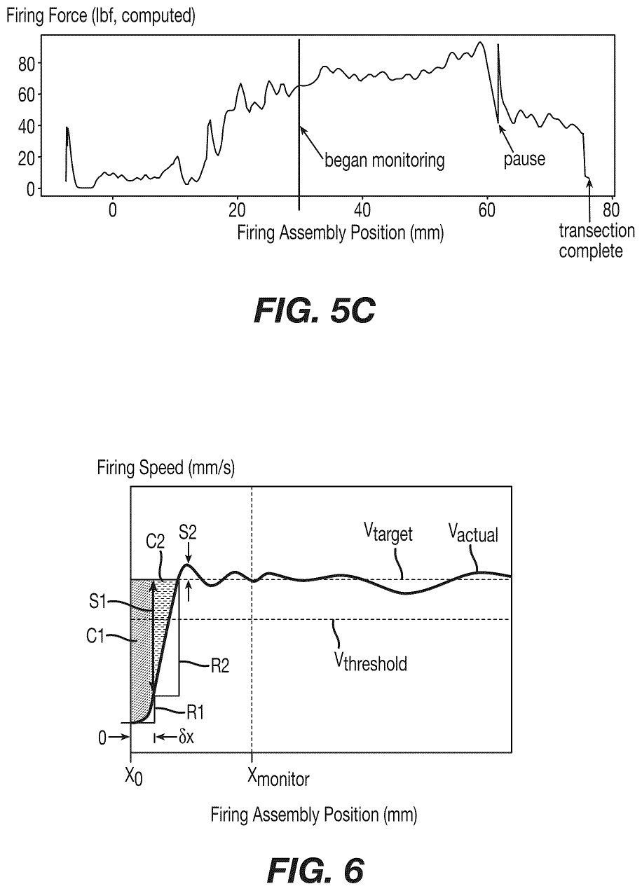

FIG. 5A is a graph of firing speed as a function of firing assembly position. In the illustrated embodiment, the pause monitoring algorithm 110 has a speed threshold of about 13 mm/s. The pause monitoring algorithm 110 (FIG. 4) is engaged when the firing assembly position is about 30 mm. The firing assembly is driven to a target speed that is greater than the speed threshold until the pause algorithm initiates a pause when the firing assembly position is about 60 mm. The firing speed drops below the threshold speed when the firing assembly position is about 60 mm. The firing assembly decelerates until reaching a paused location at about 62 mm. The speed of the firing assembly is zero at the paused location. After the pause is complete, the firing assembly is driven to a target firing speed that is greater than the threshold speed until the transection is complete.

FIG. 5B is a graph of motor power as a function of firing assembly position. The motor power is measured as a percentage of a duty cycle of a PWM signal driving the motor. In the illustrated embodiment, the pause monitoring algorithm 110 has a power threshold of about 95% duty cycle. The pause monitoring algorithm 110 (FIG. 4) is engaged when the firing assembly position is about 30 mm. The motor power is above the power threshold through the remainder of the firing stroke. Note that the pause monitoring algorithm 110 can be configured to initiate a pause when both the speed is less than the speed threshold and the power is above the power threshold or other excess power condition. In this illustrated example, the power is continuously above the power threshold. Therefore, for the purposes of this example, the pause monitoring algorithm 110 performs the same whether or not the power is monitored. The pause monitoring algorithm 110 may therefore optionally monitor power and need not be configured with a power threshold.

FIG. 5C is a graph of firing force as a function of firing assembly position for the firing stroke illustrated in FIGS. 5A and 5B. The firing force generally increases through an initial portion of the firing stroke. When the firing assembly position is about 30 mm, the pause monitoring algorithm 110 (FIG. 4) is engaged, and the firing force slowly increases with some oscillations and peaks right before the pause algorithm initiate the pause. The firing force spikes briefly after the pause is complete. After the spike, the firing force slowing decreases with some oscillations, and then quickly approaches zero at the completion of the transection.

FIG. 6 is a graph of firing speed as a function of firing assembly position, illustrating speed control variables utilized to drive the firing assembly to the target speed (V_{target}). In the illustration, the firing assembly is at an initial position (X₀), the firing speed (V_{actual}) is zero, and the target speed (V_{target}) is greater than zero. The firing speed accelerates to the target speed. At a small distance interval (δx), difference between the actual firing speed and target speed can be represented by three speed error terms: instantaneous speed error (S1), rate of change speed error (R1), and cumulative speed error (C1). The instantaneous speed error (S1) indicates a difference between the actual firing speed and the target speed at firing assembly position X₀+δx. The rate of change speed error (R1) indicates a difference between the actual firing speed at firing assembly position X₀+δx and the actual firing speed at firing assembly position X₀. The cumulative speed error (C1) indicates the integral of the difference between the actual firing speed and the target speed through a distance from X₀ to X₀+δx. Speed error terms can be utilized through some or all of the firing stroke. FIG. 6 shows second speed error terms S2, R2, C2 at a second small distance interval (δx).

In some embodiments, the motor controller 65 (FIG. 3) can be configured to drive the firing assembly to a target speed set by the control circuit 64 by using one or more of the speed error terms (FIG. 6). The motor controller 65 can be configured to dynamically adjust a duty cycle of a PWM signal (and/or current to motor) during the firing stroke to drive the firing assembly 61 to the target speed. The motor controller 65 can be configured to dynamically adjust the duty cycle (and/or current) based at least in part on an instantaneous speed error (S1, S2), a rate of change speed error (R1, R2), and/or a cumulative speed error (C1, C2).

As illustrated in FIG. 6, the actual firing speed (V_{actual}) stabilizes to approximately the target speed (V_{target}) by the time the firing assembly position has traversed an initial distance.

In some embodiments, the control circuit 64 is configured to engage the pause monitoring algorithm 110 when the firing assembly position reaches a predetermined position (Xₘₒₙᵢₜₒᵣ). The pause monitoring algorithm 110 is configured to monitor the actual firing speed (V_{actual}) and initiate a pause when the actual firing speed is below a threshold speed (V_{threshold}). During this portion of the firing stroke, the motor controller 65 continues to dynamically adjust the firing speed based on speed error terms to drive the firing assembly to the target speed.

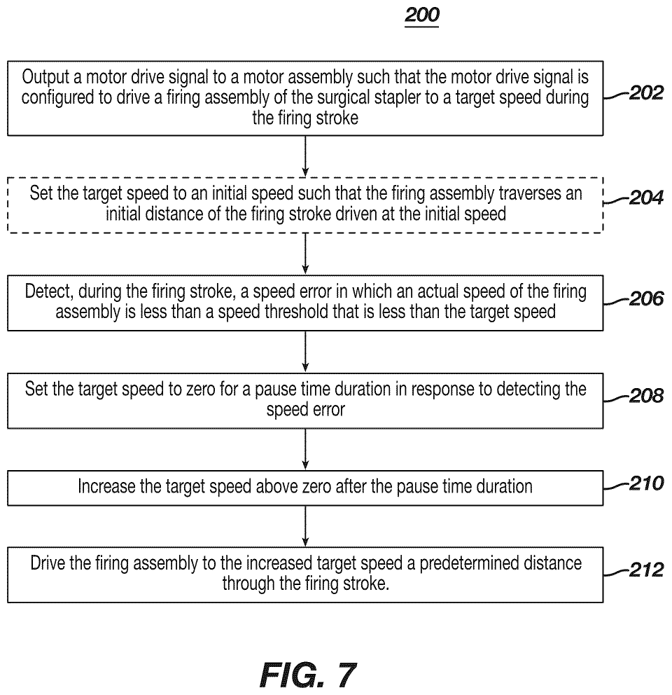

FIG. 7 is a flow diagram of a method 200 for controlling speed of a firing stroke of a surgical stapler. The method 200 may be included in a software algorithm for driving a motor of a powered surgical stapler. The method 200 is suitable for controlling speed of a firing stroke of the powered surgical stapler 10 illustrated herein, the ECHELON™ 3000 powered surgical stapler, variations thereof and alternatives thereto as understood by a person skilled in the pertinent art. The method can be carried out by the firing driver 60 illustrated herein, variations thereof and alternatives thereto as understood by a person skilled in the pertinent art. For instance, the speed control circuit 71 (FIG. 3) can include one or more processors and memory with instructions thereon, that when executed by the processor, cause the speed control circuit 71 to control speed of a firing stroke of the surgical stapler according to the method 200.

At block 202, a motor drive signal is output to a motor assembly such that the motor drive signal is configured to drive a firing assembly of the surgical stapler to a target speed during a firing stroke.

At optional block 204, the target speed can be set to an initial speed such that the firing assembly traverses an initial distance of the firing stroke driven to the initial speed.

At block 206, a speed error is detected during the firing stroke. The speed error is detected when an actual speed of the firing assembly is less than a speed threshold. The speed threshold is less than the target speed.

At block 208, a target speed is set to zero for a pause time duration in response to detecting the speed error.

At block 210, the target speed is increased to above zero after the pause time duration.

At block 212, the firing assembly is driven to the increased target speed through a predetermined distance of the firing stroke.

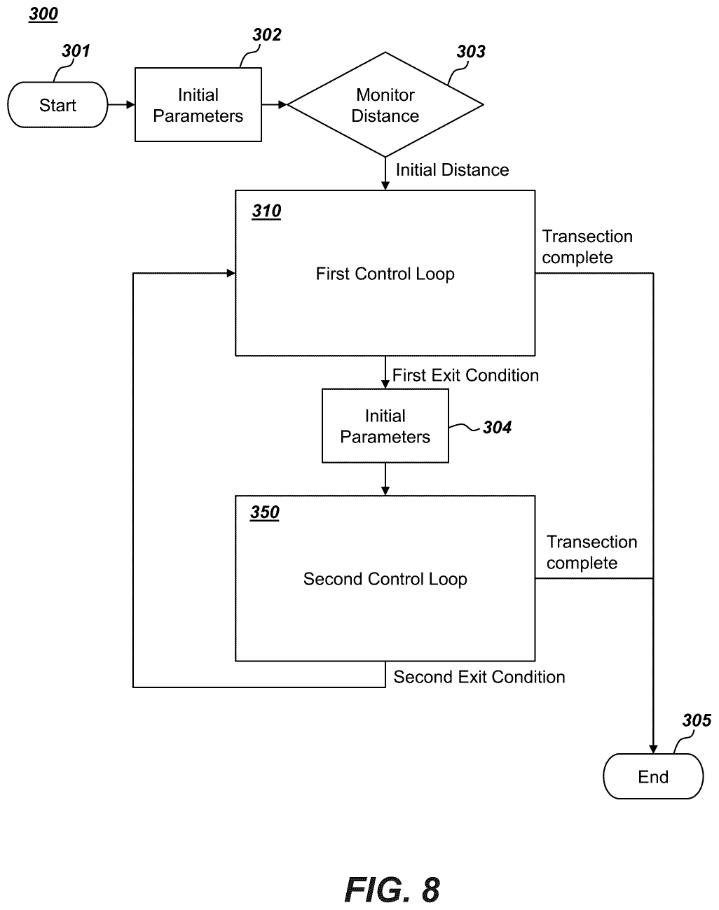

FIG. 8 is another exemplary flow diagram of a method for controlling speed of a firing stroke of a surgical stapler. FIG. 8 is an illustration of a flow diagram of an exemplary software algorithm 300 for driving a motor of a powered surgical stapler. Similar to the software algorithm 100 in FIG. 4, the software algorithm 300 is suitable for the powered surgical stapler 10 illustrated herein, the ECHELON™ 3000 powered surgical stapler, variations thereof and alternatives thereto as understood by a person skilled in the pertinent art. The software algorithm 300 can be executed by the firing driver 60 illustrated herein, variations thereof and alternatives thereto as understood by a person skilled in the pertinent art. For instance, the controller circuit 64 (FIG. 3) can include one or more processors and memory with instructions thereon, that when executed by the processor, cause the controller circuit 64 to update the motor setpoint signal output according to the software algorithm 300. This, in turn can initiate pauses and adjust the speed of a firing stroke.

At the start block 301, similar to 102 of FIG. 4, the powered surgical stapler 10 (FIG. 1) is ready to proceed with a firing stroke. A knife 43, I-beam 45, or other distal portion of a firing assembly 61 may be at a home position X0 in the end effector 40 (FIGS. 2 and 3).

At block 302, the software algorithm 300 provides initial parameters. The initial parameters may include for example, an initial target firing speed, an initial power threshold, and/or an initial distance. As discussed above, in some embodiments, the initial target firing speed is approximately 12 mm/s to approximately 16 mm/s, and more preferably at approximately 12 mm/s. The initial power threshold may be statically predefined or assigned to the surgical stapler 10 via an instrument-specific characterization process. Power to a motor driving the firing assembly (e.g., motor 63 driving firing assembly 61) and the power threshold can be based on duty cycle of a PWM signal driving the motor, current draw by the motor, or other suitable parameter as understood by a person skilled in the pertinent art. The initial power threshold can be a percentage of total available power, e.g., duty cycle percentage, or a percentage of a maximum current rating of the motor. In some embodiments, the initial power threshold is based on a typical use case condition with normal or thin tissue with sufficient margin so that the power threshold can be increased during the firing stroke to adapt to thicker tissue conditions. In some embodiments, the initial power threshold is between 50% and 70%.

At block 303, similar to block 106 of FIG. 4, the software algorithm 300 maintains the initial target firing speed while monitoring distance of travel of the firing assembly 61. The software algorithm 300 determines the firing assembly 61 is at or beyond the initial distance and proceeds to block 310. In some embodiments, the speed control circuit 71 is configured to output a motor drive signal to the motor assembly to drive the firing assembly to the target speed through the initial distance of the firing stroke. The speed control circuit 71 dynamically adjusts the power to the motor assembly in an attempt to reach and maintain the target speed. During the initial distance the power to the motor assembly may surpass the initial target threshold while the speed control circuit continues to dynamically adjust the power to the motor assembly to achieve the target speed. Likewise, the actual speed may vary beyond a speed error while the speed control circuit continues to dynamically adjust the power to the motor assembly to achieve the target speed. In some embodiments, there is no execution of the first control loop 310 or the second control loop 350 during the initial distance. In other words, a detection of a speed error or an excess power condition, which would be discussed below, is not executed as the firing assembly is moved through initial distance.

At block 310, the software algorithm 300 executes a first control loop during the firing stroke. When the firing assembly 61 travels beyond the initial distance, a motor drive signal can be output to the motor assembly to continue to dynamically adjust power to the motor assembly throughout a firing stroke. The first control loop can include an algorithm which initiates a pause during the firing stroke in response to an occurrence of an excess power condition. The excess power condition refers to a scenario wherein an actual power to the motor assembly is greater than a power threshold. The first control loop can repeatedly pause and restart based on repeated occurrence of an excess power condition. A first pause of the first loop can be triggered by the actual power to the motor assembly exceeding the initial power threshold. In some embodiments, the power threshold can be adjusted following each pause in the first loop.

As discussed elsewhere herein, during a pause, the target speed is set to zero and the firing assembly decelerates and stops. The pause may increase localized compression of tissue between the jaws of the end effector. The pause can last for a pause duration which is long enough to allow the tissue to relax, which reduces strain on the jaws of the end effector and the firing assembly system. In some examples, such pause may be one second.

The algorithm 300 is configured exit the first loop upon completion of the transection (in which case the method 300 proceeds to end block 305). The software algorithm 300 is also configured to exit the first control loop in response to a first exit condition being met. In some embodiments, the first exit condition is met when the power threshold is increased to or beyond a maximum power threshold after an occurrence of an excess power condition. Additionally, or alternatively, the first exit condition is met when a number of pauses initiated during the first control loop 310 reaches a pause count threshold. In some embodiments, the number of pauses initiated can be counted by counting the number of times the target speed is set to zero and held at zero for some predetermined amount of time. The duration of each pause in the first loop is referred to herein as “a first pause duration”. Note that the first loop may execute more than one pause, each lasting for the first pause duration respectively. When the first control loop 310 is exited due to the first exit condition being met, the algorithm 300 proceeds to block 304.

At block 304, the software algorithm 300 provides initial parameters as needed for execution of a second control loop (block 350). For instance, an initial speed error threshold can be set. The speed error threshold can be based on factors disclosed elsewhere herein, including but not limited to the speed error terms illustrated in FIG. 6.

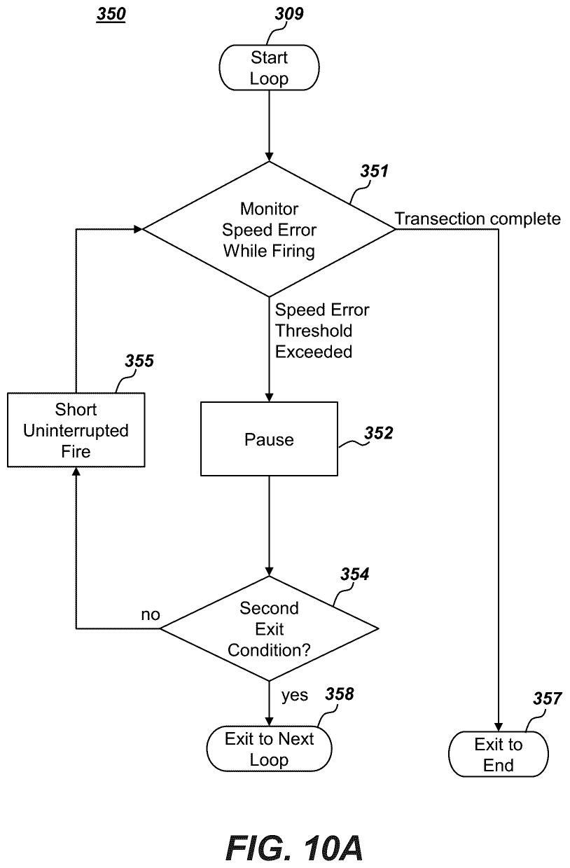

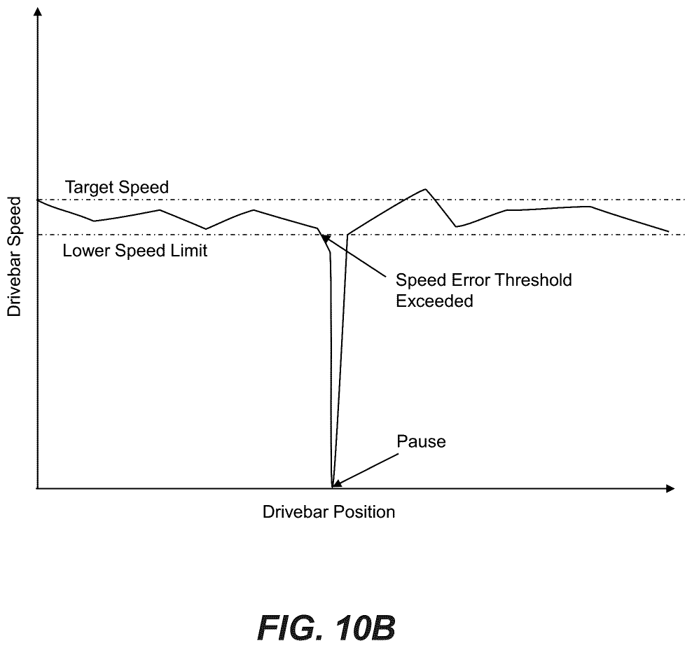

At block 350, the software algorithm 300 executes the second control loop during the firing stroke in which a pause is initiated during the firing stroke in response to a detection of a speed error condition. In some embodiments, the speed error condition is met when an actual speed of the firing assembly 61 is less than a speed threshold (e.g., the “lower speed limit” in FIG. 10B), wherein the speed threshold is less than a target speed. The software algorithm 300 monitors the actual speed of the firing assembly and detects the speed error condition. The speed error condition can additionally or alternatively be based on factors disclosed elsewhere herein, including but not limited to the speed error terms illustrated in FIG. 6.

The second control loop can repeatedly pause and restart based on repeated occurrence of an speed error. A first pause of the second loop can be triggered by the actual speed of the motor and/or firing assembly exceeding the initial speed error threshold set at block 304. In some embodiments, the speed error threshold can be adjusted following each pause in the second loop.

As discussed elsewhere herein, during a pause, the target speed is set to zero and the firing assembly decelerates and stops. The duration of each pause in the second loop is referred to herein as “a second pause duration”. Note that the second loop may execute more than one pause, each lasting for the second pause duration respectively.

The algorithm 300 is configured to exit the second loop upon completion of the transection (in which case the method 300 proceeds to end block 305). The software algorithm 300 is also configured to exit the second control loop in response to a second exit condition being met. In some embodiments, the second exit condition is not based on the number of pauses which are initiated during the second control loop. In such embodiments, the algorithm 300 may pause knife (firing assembly) movement an unlimited quantity of times in the second control loop. The second exit condition can be predefined and/or based on a measured device individual characteristic. In an alternative embodiment the second loop can exit in response to a pause count threshold being met.

Upon exiting the second control loop due to the second exit condition being met, the algorithm 300 reenters the first control loop at block 310. Upon reentry of the first control loop, the excess power condition can be based on initial parameters set at block 302. For instance, the power threshold upon reentry of the first control loop can be reset to the initial power threshold set at block 302.

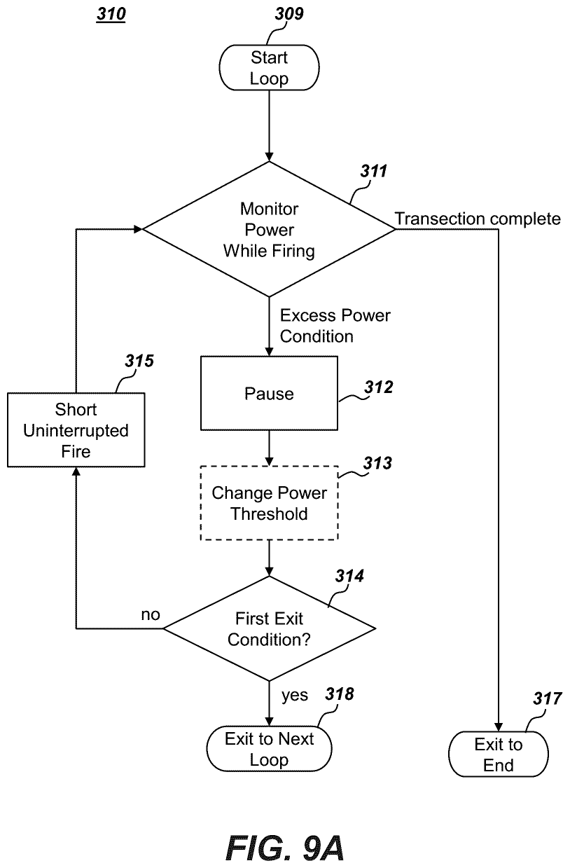

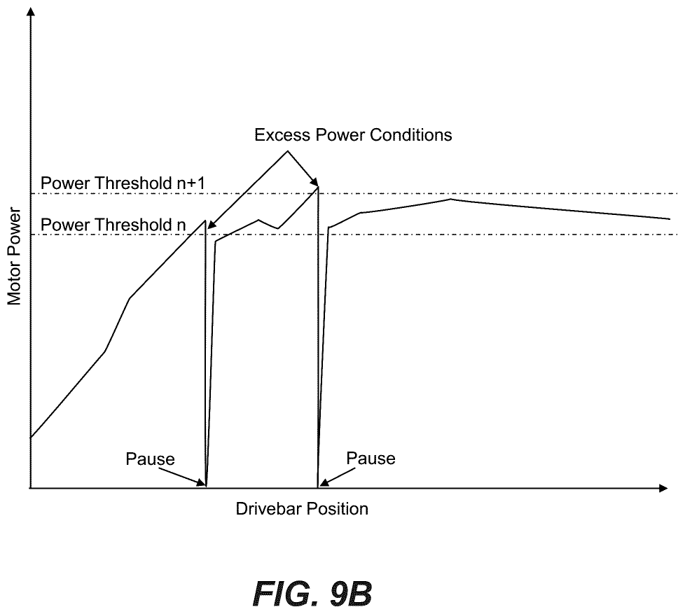

FIG. 9A is an exemplary flow diagram illustrating a pausing monitoring process executed within the first control loop 310 in FIG. 8.

At block 309, the first control loop 310 starts in response to the firing assembly 61 travelling to or beyond an initial distance (i.e., from block 303 in FIG. 8) or in response to a second exit condition being met in the second control loop (i.e., from block 350).

At block 311, the software algorithm 300, or alternatively another software algorithm, monitors an actual power of the motor assembly during the firing stroke. The power may be monitored by measuring or monitoring a duty cycle of the motor assembly. Completion of the transection can also be detected at block 311. When transection is complete, the first control loop 310 exits at block 317 to end block 305 (FIG. 8). In the first control loop 310, the target speed may vary significantly without triggering an excess power condition. For instance, the target speed may surpass a speed error condition set at blocks 302 or 304 of the algorithm 300 (FIG. 8) without triggering an excess power condition. In some embodiments, the excess power condition is the sole condition which must be met to cause the first control loop 310 to pause (i.e., proceed to block 312).

If an excess power condition occurs or is detected during the firing stroke, the first control loop 310 proceeds to block 312 and a pause of the firing assembly is initiated. The excess power condition can be met when actual power of the motor assembly exceeds a power threshold. As discussed above, in some embodiments, the motor drive signal comprises a PWM signal. The duty cycle can be dynamically adjusted during the firing stroke so that the firing assembly is driven to the target speed. A high duty cycle may be required when more force is required on the firing assembly to achieve the target speed. In such embodiments, the excess power condition can be detected by measuring a duty cycle of the PWM signal. In some embodiments, the power threshold comprises a predetermined duty cycle percentage value. In this scenario, the detection of the excess power condition is a detection of a duty cycle of the PWM signal being greater than a predetermined duty cycle percentage value. The power threshold can alternatively be based on motor current draw or other excess power condition as disclosed elsewhere herein and understood by a person skilled in the pertinent art.

At block 312, the target speed is set to zero during the first pause time duration. The pause of block 312 can be executed according to pausing disclosed in more detail elsewhere herein, for instance, in relation to block 310 of FIG. 8 and block 112 of FIG. 4.

At block 313, optionally, before resuming the firing stroke or the traveling of the firing assembly during the firing stroke, the power threshold is changed. Such change may be performed during the first pause time duration, or right after the pause. In some embodiments, the power threshold is increased as illustrated in FIG. 9B. In some embodiments, the power threshold comprises a predetermined duty cycle percentage value. The target speed is increased to a value above zero after the pause time duration.

As discussed above (e.g., block 113 of FIG. 4), optionally, the target firing speed may be updated, which for example may be less than the initial target firing speed or the target firing speed prior to the specific pause at block 312. It is to be understood, after the first pause time duration, the process resumes driving the firing assembly to the increased (e.g., updated) target speed during the firing stroke. In some embodiments, the firing assembly is driven to the increased target speed a predetermined distance through the firing stroke following the pause executed at block 312.

At block 314, a determination is made as for whether a first exit condition is being met. As discussed above, the first exit condition may be met when a number of pauses initiated within the first control loop reaches a first pause count threshold, and/or when the power threshold is at or exceeds a maximum power threshold. The power threshold may be at or exceed the maximum power threshold due to an increase of the power threshold at block 313.

When the first exit condition is met at block 314, the first control loop 310 proceeds to block 318. Otherwise, the first control loop 310 proceeds to block 315.

At block 318, the process exits the first control loop 310 and proceeds to next loop, i.e., the second control loop 350 which is described in conjunction with FIGS. 8 and 10A.

At block 315, similar to block 115 in FIG. 4, the first control loop 310 may initiate a short uninterrupted firing stroke portion in which the firing assembly travels a predetermined distance without the process monitoring the actual power and determining whether an excess power condition occurs or exist. That is to say, the detection of the excess power condition is disabled and therefore a subsequent pause will not be initiated at least until the firing assembly has traveled the predetermined distance. The predetermined distance can be set based on the distance from the firing assembly 61 or the location of the firing assembly 61 during the firing stroke. For example, the predetermined distance can be set based on a position of the firing assembly where the target speed is set to zero during the most recent pause. The predetermined distance may otherwise be set as disclosed elsewhere herein. Upon completion of block 315, the first control loop 310 proceeds again to block 311.

FIG. 9B is a graph in which motor power is plotted against drivebar position during a portion of a firing stroke in which the first control loop 310 is executed in an exemplary scenario. As illustrated in FIG. 9B, the first control loop 310 is executed with a power threshold of Power Threshold n. The actual power increases until it exceeds the Power Threshold n. An excess power condition occurs and is detected at block 311 of the first control loop 310 (FIG. 9A). As discussed above, the power threshold can be changed at block 313 in FIG. 9A. FIG. 9B illustrates a scenario in which the power threshold is increased to Power Threshold n+1 after a pause is triggered. The increase can occur during the pause or after completion of the short uninterrupted fire, so long as the increase occurs before reentry to block 311 (FIG. 9A). In some embodiments, the power threshold may be increased by a predetermined value each time. In some further embodiments, a maximum threshold is set, which can be a condition of the first exit condition as discussed above. As illustrated in FIG. 9B, the motor power increases following the first pause, surpassing the previous power threshold (Power Threshold n) without occurrence of an excess power condition. The motor power continues to increase through the firing stroke until it exceed the increased power threshold (Power Threshold n+1). An excess power condition is again detected at block 311 of the first control loop 310 (FIG. 9A). The first control loop initiates another pause, and the firing stroke resumes after completing of a first pause duration. Although not shown, the power threshold can be increased a second time following the second pause. Although not shown, the power threshold can be increased after multiple pauses until the power threshold is at a maximum threshold and there is no more power available from the motor drive assembly.

FIG. 10A is an exemplary flow diagram illustrating a pausing monitoring process executed within the second control loop 350 in FIG. 8.

At block 309, the process enters the second control loop 350 in response to the exiting of the first control loop 310 (FIG. 8) due to the first exit condition being met.

At block 351, the process executed by the software algorithm 300, or alternatively another software algorithm, monitors an actual speed of the firing assembly 61 and detects a speed error. Completion of the transection can also be detected at block 351. When transection is complete, the second control loop 350 exits at block 357 to end block 305 (FIG. 8). In the second control loop 350, the actual speed to the motor assembly may vary significantly without triggering a speed error. For instance, the power (e.g., as determined by current and/or duty cycle measurement) may surpass a power threshold set at block 302 or during the first control loop 310 of the algorithm 300 (FIG. 8) without triggering a speed error. In some embodiments, the detection of a speed error is the sole condition which must be met to cause the second control loop 350 to pause (i.e., proceed to block 352).

In some embodiments the speed error refers to a scenario in which the actual speed of the firing assembly is less than a speed threshold that is less than the target speed as illustrated in FIG. 10B. FIG. 10B is a graph of an initiated pause and a change of target speed during a portion of a firing stroke. In FIG. 10B, when a monitored actual speed is less than the Lower Speed Limit, a speed error threshold is exceeded and a speed error will be detected by the process. Additionally, or alternatively, the speed error can be detected as disclosed elsewhere herein, for instance as described in relation to block 310 of FIG. 8, as described in relation to block 111 of FIG. 4, and using any combination of parameters described in relation to FIG. 6.

At block 352, in response to each time a speed error is detected, the process initiates a pause for a second pause time duration, during which the target speed is set to zero. The pause of block 352 can be executed according to pausing disclosed in more detail elsewhere herein, for instance, in relation to block 350 of FIG. 8 and block 112 of FIG. 4.

At block 354, a determination is made as for whether a second exit condition being met. When the second exit condition is met, the second control loop 350 proceeds to block 358. Otherwise, the second control loop 350 proceeds to block 355.

At block 358, the process exits the second control loop 350 and proceeds to next loop, for example, reentering the first control loop 310 as described in relation to FIG. 8.

At block 355, Similar to block 315 in FIG. 9A and block 115 in FIG. 4, the second control loop 350 can initiate a short uninterrupted firing stroke portion in which the firing assembly travels a predetermined distance without the process monitoring the actual speed and determining whether a speed error occurs or exists. That is to say, the detection of the speed error is disabled and therefore a subsequent pause will not be initiated at least until the firing assembly has traveled the predetermined distance.

The predetermined distance may be set based on the distance from the firing assembly 61 or the location of the firing assembly 61 during the firing stroke. For example, the predetermined distance can be set based on a position of the firing assembly where the target speed is set to zero during the most recent pause. The predetermined distance may otherwise be set as disclosed elsewhere herein. Upon completion of block 315, the first control loop 310 proceeds again to block 311.

FIG. 10B is a graph in which drivebar speed (i.e., firing assembly speed) is plotted against drivebar position during a portion of a firing stroke in which a second, speed-based, control loop 350 is executed. The firing assembly is driven to the target speed by dynamically adjusting power to the motor assembly. The actual speed oscillates due to variations in force on the firing assembly during the firing stroke and the control circuit attempts to compensate by adjusting power to the motor. A speed error is detected when a speed error threshold is exceeded, i.e., the drivebar speed is less than a lower speed limit (FIG. 10A block 351). After the pause, the second control loop 350 resumes the driving the firing assembly through the firing stroke according to the algorithm of the second control loop 350.

In some embodiments, related parameters such as the duty cycle, speed threshold, the initial distance, the disabled zone in which related detection or monitor is disabled, or any appropriate parameters illustrated above in this disclosure, may be statically predefined or assigned in the system (e.g., via an instrument-specific characterization process). For example, the disabled zone can be defined through characterization to positions along a drivebar stroke, which is independent of the pause actuation.

In some embodiments, any appropriate monitored or set parameters, such as any of the actual power, the power threshold, the target speed, the actual current speed, the historical or current speed error, or the increased target speed, or the increased power threshold mentioned above, can be transmitted to and displayed at a console or display. For example, the related data can be transmitted directly or indirectly (e.g., wirelessly to a central “hub” or router) for a display.

The following clauses list non-limiting embodiments of the disclosure:
Clause 1. A surgical stapler comprising: a firing assembly configured to translate along a longitudinal axis such that translation of the firing assembly in a distal direction is configured to deploy staples from an end effector; a motor assembly mechanically coupled to the firing assembly and configured to drive the firing assembly along the longitudinal axis; and a speed control circuit configured to: output a motor drive signal to the motor assembly such that the motor drive signal is configured to drive the firing assembly to a target speed during a firing stroke, detect, during the firing stroke, a speed error in which an actual speed of the firing assembly is less than a speed threshold that is less than the target speed, set the target speed to zero for a pause time duration in response to detecting the speed error, increase the target speed above zero after the pause time duration, and drive the firing assembly to the increased target speed a predetermined distance through the firing stroke.
Clause 2. The surgical stapler of clause 1, wherein the motor drive signal comprises a pulse width modulated (PWM) signal.
Clause 3. The surgical stapler of clause 2, wherein the speed control circuit is configured to dynamically adjust a duty cycle of the PWM signal during the firing stroke to drive the firing assembly to the target speed.
Clause 4. The surgical stapler of clause 2 or 3, wherein the speed control circuit is configured to: detect, during the firing stroke, an excess power condition in which the duty cycle of the PWM signal is greater than a duty cycle threshold and set the target speed to zero for the pause time duration in response to detecting the excess power condition.
Clause 5. The surgical stapler of any one of clauses 2-4, wherein the speed control circuit is configured to dynamically adjust the duty cycle of the PWM signal based at least on an instantaneous speed error, a rate of change speed error, or a cumulative speed error.
Clause 6. The surgical stapler of any one of clauses 2-5, wherein the speed control circuit is configured to dynamically adjust the duty cycle of the PWM signal based at least on an instantaneous speed error measured as an instantaneous difference between the actual speed and the target speed, the instantaneous speed error being greater than a difference between the target speed and the speed threshold.
Clause 7. The surgical stapler of any one of clauses 1-6, wherein the speed control circuit is further configured to count a number of pause time durations during the firing stroke and drive the firing assembly to the increased target speed through completion of the firing stroke when the number of pause time durations is above a pause count threshold.
Clause 8. The surgical stapler of any one of clauses 1-7, wherein the speed control circuit is configured to: set the target speed to an initial speed such that the firing assembly traverses an initial distance of the firing stroke driven to the initial speed.
Clause 9. The surgical stapler of clause 8, wherein the speed control circuit is configured to: set the target speed after the pause time duration such that the target speed is set less than the initial speed and greater than zero.
Clause 10. The surgical stapler of clause 8 or 9, wherein the initial speed is approximately 12 mm/s to approximately 16 mm/s.
Clause 11. The surgical stapler of any one of clauses 8-10, wherein the initial speed is approximately 12 mm/s.
Clause 12. The surgical stapler of any one of clauses 8-11, wherein the motor drive signal is a PWM signal having less than 100% duty cycle in an unloaded firing condition as the firing assembly travels at the initial speed.
Clause 13. The surgical stapler of any one of clauses 1-12, wherein the pause time duration is approximately one second.
Clause 14. The surgical stapler of any one of clauses 1-13, further comprising: one or more position sensors; and a timer, wherein the speed control circuit is configured to determine the actual speed of the firing assembly based at least in part on electrical signals from the one or more position sensors and the timer.
Clause 15. The surgical stapler of any one of clauses 1-14, wherein the speed control circuit is configured to: detect, during the firing stroke, the speed error when the actual speed of the firing assembly is less than the speed threshold that is less than the target speed and electrical current driving the motor assembly is greater than a current threshold.
Clause 16. The surgical stapler of any one of clauses 1-15, wherein the firing assembly comprises a firing bar, a knife, an I-beam, a wedge sled, or any combination thereof.
Clause 17. The surgical stapler of any one of clauses 1-16, wherein the end effector comprises an anvil and a staple jaw, and wherein the firing assembly comprises an I-beam configured to engage the anvil and staple jaw during the firing stroke.
Clause 18. A method for controlling speed of a firing stroke of a surgical stapler, the method comprising: outputting a motor drive signal to a motor assembly such that the motor drive signal is configured to drive a firing assembly of the surgical stapler to a target speed during the firing stroke; detecting, during the firing stroke, a speed error in which an actual speed of the firing assembly is less than a speed threshold that is less than the target speed; setting the target speed to zero for a pause time duration in response to detecting the speed error; increasing the target speed above zero after the pause time duration; and driving the firing assembly to the increased target speed a predetermined distance through the firing stroke.
Clause 19. The method of clause 18, wherein the motor drive signal comprises a pulse width modulated (PWM) signal.
Clause 20. The method of clause 19, wherein outputting the motor drive signal to the motor assembly such that the motor drive signal is configured to drive the firing assembly of the surgical stapler to the target speed during the firing stroke comprises dynamically adjusting a duty cycle of the PWM signal during the firing stroke to drive the firing assembly to the target speed.
Clause 21. The method of clause 19 or 20, comprising: detecting, during the firing stroke, an excess power condition in which the duty cycle of the PWM signal is greater than a duty cycle threshold and setting the target speed to zero for the pause time duration in response to detecting the excess power condition.
Clause 22. The method of any one of clauses 19-21, comprising: dynamically adjusting the duty cycle of the PWM signal based at least on an instantaneous speed error, a rate of change speed error, or a cumulative speed error.
Clause 23. The method of any one of clauses 19-22, comprising: dynamically adjust the duty cycle of the PWM signal based at least on an instantaneous speed error measured as an instantaneous difference between the actual speed and the target speed, the instantaneous speed error being greater than a difference between the target speed and the speed threshold.
Clause 24. The method of any one of clauses 18-23, comprising: counting a number of pause time durations during the firing stroke; and driving the firing assembly to the increased target speed through completion of the firing stroke when the number of pause time durations is above a pause count threshold.
Clause 25. The method of any one of clauses 18-24, comprising: setting the target speed to an initial speed such that the firing assembly traverses an initial distance of the firing stroke driven to the initial speed.
Clause 26. The method of clause 25, wherein increasing the target speed above zero after the pause time duration comprises setting the target speed less than the initial speed and greater than zero after the pause time duration.
Clause 27. The method of clause 25 or 26, wherein the initial speed is approximately 12 mm/s to approximately 16 mm/s.
Clause 28. The method of any one of clauses 25-27, wherein the initial speed is approximately 12 mm/s.
Clause 29. The method of any one of clauses 25-28, wherein the motor drive signal is a PWM signal having less than 100% duty cycle in an unloaded firing condition as the firing assembly travels at the initial speed.
Clause 30. The method of any one of clauses 18-29, wherein the pause time duration is approximately one second.
Clause 31. The method of any one of clauses 18-29, comprising: determine the actual speed of the firing assembly based at least in part on electrical signals from one or more position sensors and a timer of the surgical stapler.
Clause 32. The method of any one of clauses 18-31, comprising: detecting, during the firing stroke, the speed error when the actual speed of the firing assembly is less than the speed threshold that is less than the target speed and electrical current driving the motor assembly is greater than a current threshold.
Clause 33. A method for controlling speed of a firing stroke of a surgical stapler, the method comprising: outputting a motor drive signal to a motor assembly such that the motor drive signal is configured to drive a firing assembly of the surgical stapler to a target speed during the firing stroke; detecting, during the firing stroke, a speed error in which an actual speed of the firing assembly is less than a speed threshold that is less than the target speed and a parameter of the motor drive signal is beyond a threshold value; setting the target speed to zero for a pause time duration in response to detecting the speed error; increasing the target speed above zero after the pause time duration; and driving the firing assembly to the increased target speed a predetermined distance through the firing stroke.
Clause 34. The method of clause 33, comprising: wherein the motor drive signal comprises a pulse width modulated (PWM) signal, and wherein the parameter of the motor drive signal comprises a duty cycle of the PWM signal.
Clause 35. The method of clause 33 or 34, wherein the parameter of the motor drive signal comprises a magnitude of electrical current driving the motor assembly.
Clause 36. The surgical stapler of any one of clauses 1-17, wherein the speed control circuit is configured to: dynamically adjust power to the motor assembly to drive the firing assembly to the target speed during the firing stroke; execute a first control loop during the firing stroke in which the speed control circuit is configured to monitor an actual power of the motor assembly and set the target speed to zero for the pause time duration each time an excess power condition occurs; and execute a second control loop during the firing stroke in which the speed control circuit is configured to monitor the actual speed of the firing assembly and set the target speed to zero for the pause time duration each time the speed error is detected.
Clause 37. The method of any one of clauses 18-35, comprising: dynamically adjusting power to the motor assembly to drive the firing assembly to the target speed during the firing stroke; executing a first control loop during the firing stroke in which an actual power of the motor assembly is monitored and the target speed is set to zero for the pause time duration each time an excess power condition occurs; and executing a second control loop during the firing stroke in which the actual speed of the firing assembly is monitored and the target speed is set to zero for the pause time duration each time the speed error is detected.
Clause 38. A surgical stapler comprising: a firing assembly configured to translate along a longitudinal axis such that translation of the firing assembly in a distal direction is configured to deploy staples from an end effector; a motor assembly mechanically coupled to the firing assembly and configured to drive the firing assembly along the longitudinal axis; and a speed control circuit configured to: output a motor drive signal to the motor assembly such that the motor drive signal is configured to dynamically adjust power to the motor assembly to drive the firing assembly to a target speed during a firing stroke, detect, during the firing stroke, an excess power condition in which an actual power to the motor assembly is greater than a power threshold, set the target speed to zero for a first pause time duration in response to detecting the excess power condition, increase the target speed above zero after the first pause time duration and increase the power threshold before resuming the fire stroke, and resume driving the firing assembly to the increased target speed during the firing stroke.
Clause 39. The surgical stapler of clause 38, wherein the speed control circuit is configured to disable the detection of the excess power condition during driving the firing assembly to the increased target speed a predetermined distance through the firing stroke.
Clause 40. The surgical stapler of clause 39, wherein the predetermined distance is set based on a position of the firing assembly where the target speed is set to zero.
Clause 41. The surgical stapler of any one of clauses 38-40, wherein the speed control circuit is configured to drive the firing assembly to the increased target speed a predetermined distance through the firing stroke following the first pause time duration.
Clause 42. The surgical stapler of any one of clauses 38-42, wherein the motor drive signal comprises a pulse width modulated (PWM) signal.
Clause 43. The surgical stapler of clause 42, wherein the speed control circuit is configured to detect the excess power condition by measuring a duty cycle of the PWM signal.
Clause 44. The surgical stapler of clause 43, wherein the power threshold comprises a predetermined duty cycle percentage value.
Clause 45. The surgical stapler of any one of clauses 38-44, wherein the speed control circuit is configured to increase the power threshold by a predetermined value.
Clause 46. The surgical stapler of any one of clauses 38-45, wherein, in response to the power threshold being increased to a maximum power threshold, the speed control circuit is configured to: disable the detection of the excess power condition, detect, during the firing stroke, a speed error in which an actual speed of the firing assembly is less than a speed threshold that is less than the target speed, set the target speed to zero for a second pause time duration in response to detecting the speed error, and increase the target speed above zero after the second pause time duration.
Clause 47. The surgical stapler of any one of clauses 38-45, wherein, in response to a number of setting the target speed to zero for the first pause time duration reaching a first pause count threshold, the speed control circuit is configured to: disable the detection of the excess power condition, detect, during the firing stroke, a speed error in which an actual speed of the firing assembly is less than a speed threshold that is less than the target speed, set the target speed to zero for a second pause time duration in response to detecting the speed error, and increase the target speed above zero after the second pause time duration.
Clause 48. The surgical stapler of any one of clauses 46-47, in response to a measurement of the surgical stapler meeting a predetermined condition, the speed control circuit is configured to: disable the detection of the speed error; set the power threshold to a minimum predetermined value; and resume the detection of the excess power condition.
Clause 49. The surgical stapler of any one of clauses 38-48, wherein the speed control circuit is configured to disable the detection of the excess power condition during an initial distance at a beginning of the firing stroke.
Clause 50. The surgical stapler of any one of clauses 38-49, wherein at least one of the actual power level, the power threshold, the target speed, the increased target speed, or the increased power threshold is transmitted to and displayed at a console or display.
Clause 51. A method for controlling speed of a firing stroke of a surgical stapler, the method comprising: outputting a motor drive signal to a motor assembly such that the motor drive signal is configured to dynamically adjust power to the motor assembly to drive a firing assembly to a target speed during a firing stroke, detecting, during the firing stroke, an excess power condition in which an actual power to the motor assembly is greater than a power threshold, setting the target speed to zero for a first pause time duration in response to detecting the excess power condition, increasing the target speed above zero after the first pause time duration and increasing the power threshold before resuming the fire stroke, and resuming driving the firing assembly to the increased target speed during the firing stroke.
Clause 52. The method of clause 51, comprising: disabling the detection of the excess power condition during driving the firing assembly to the increased target speed a predetermined distance through the firing stroke.
Clause 53. The method of clause 52, comprising: setting the predetermined distance based on a position of the firing assembly where the target speed is set to zero.
Clause 54. The method of any one of clauses 51-53, comprising: driving the firing assembly to the increased target speed a predetermined distance through the firing stroke following the first pause time duration.
Clause 55. The method of any one of clauses 51-54, wherein the motor drive signal comprises a pulse width modulated (PWM) signal.
Clause 56. The method of clause 55, wherein detecting the excess power condition comprises measuring a duty cycle of the PWM signal.
Clause 57. The method of clause 56, wherein the power threshold comprises a predetermined duty cycle percentage value.
Clause 58. The method of any one of clauses 51-57, comprising: increasing the power threshold by a predetermined value before resuming the fire stroke.
Clause 59. The method of any one of clauses 51-58, comprising: disabling, in response to the power threshold being increased to a maximum power threshold, the detection of the excess power condition; detecting a speed error in which an actual speed of the firing assembly is less than a speed threshold that is less than the target speed; setting the target speed to zero for a second pause time duration in response to detecting the speed error; and increasing the target speed above zero after the second pause time duration.
Clause 60. The method of any one of clauses 51-58, comprising: disabling, in response to a number of setting the target speed to zero for the first pause time duration reaching a first pause count threshold, the detection of the excess power condition; detecting a speed error in which an actual speed of the firing assembly is less than a speed threshold that is less than the target speed; setting the target speed to zero for a second pause time duration in response to detecting the speed error; and increasing the target speed above zero after the second pause time duration.
Clause 61. The method of clause 59 or 60, comprising: disabling, in response to a measurement of the surgical stapler meeting a predetermined condition, the detection of the speed error; setting the power threshold to a minimum predetermined value; and resuming the detection of the excess power condition.
Clause 62. The method of any one of clauses 51-61, comprising: disabling the detection of the excess power condition during an initial distance at a beginning of the firing stroke.
Clause 63. The method of any one of clauses 51-62, comprising: transmitting at least one of the actual power level, the power threshold, the target speed, the increased target speed, or the increased power threshold to a display.
Clause 64. A surgical stapler comprising: a firing assembly configured to translate along a longitudinal axis such that translation of the firing assembly in a distal direction is configured to deploy staples from an end effector; a motor assembly mechanically coupled to the firing assembly and configured to drive the firing assembly along the longitudinal axis; and a speed control circuit configured to: output a motor drive signal to the motor assembly such that the motor drive signal is configured to dynamically adjust power to the motor assembly to drive the firing assembly to a target speed during a firing stroke, execute a first control loop during the firing stroke in which the speed control circuit is configured to initiate a pause during the firing stroke in response to a detection of an excess power condition in which an actual power to the motor assembly is greater than a power threshold, and execute a second control loop during the firing stroke in which the speed control circuit is configured to initiate a pause during the firing stroke in response to a detection of a speed error condition in which an actual speed of the firing assembly is less than a speed threshold that is less than the target speed, wherein the second control loop is distinct from the first control loop.
Clause 65. The surgical stapler of clause 64, wherein the speed control circuit is configured to: exit the first control loop in response to a first exit condition being met.
Clause 66. The surgical stapler of clause 65, wherein the first exit condition is met when a number of pauses initiated by the first control loop reaches a first pause count threshold.
Clause 67. The surgical stapler of clause 64 or 65, wherein the first exit condition is met when the power threshold is at a maximum power threshold and the excess power condition occurs.
Clause 68. The surgical stapler of any one of clauses 65-67, wherein the speed control circuit is configured to: enter the second control loop in response to the exiting of the first control loop due to the first exit condition being met.
Clause 69. The surgical stapler of clause 68, wherein the speed control circuit is configured to: exit the second control loop in response to a second exit condition being met; and reenter the first control loop in response to exiting of the second control loop in response to the second exit condition being met.
Clause 70. The surgical stapler of any one of clauses 64-69, wherein the excess power condition occurs when the actual power of the motor assembly exceeds a power threshold, wherein, while executing the first control loop, the speed control circuit is configured to increase the power threshold during a pause.
Clause 71. The surgical stapler of any one of clauses 64-70, wherein the speed control circuit is configured to: exit the first and/or second control loop upon completion of the firing stroke.
Clause 72. The surgical stapler of any one of clauses 64-71, wherein, the control circuit is configured to drive the firing assembly a predetermined distance through the firing stroke following a pause initiated in the first control loop such that a subsequent pause is not initiated at least until the firing assembly has traveled the predetermined distance.
Clause 73. The surgical stapler of any one of clauses 64-72, wherein, the control circuit is configured to drive the firing assembly to a predetermined distance through the firing stroke following a pause initiated in the second control loop such that a subsequent pause is not initiated at least until the firing assembly has traveled the predetermined distance.
Clause 74. The surgical stapler of any one of clauses 64-73, wherein the motor drive signal comprises a pulse width modulated (PWM) signal.
Clause 75. The surgical stapler of clause 74, wherein the control circuit is configured to detect the excess power condition in which a duty cycle of the PWM signal is greater than a predetermined duty cycle percentage value.
Clause 76. A method for controlling speed of a firing stroke of a surgical stapler, the method comprising: outputting a motor drive signal to a motor assembly such that the motor drive signal is configured to dynamically adjust power to the motor assembly to drive a firing assembly to a target speed during a firing stroke; executing a first control loop during the firing stroke in which a pause is initiated during the firing stroke in response to a detection of an excess power condition in which an actual power to the motor assembly is greater than a power threshold; and executing a second control loop during the firing stroke in which a pause is initiated during the firing stroke in response to a detection of a speed error condition in which an actual speed of the firing assembly is less than a speed threshold that is less than the target speed, wherein the second control loop is distinct from the first control loop.
Clause 77. The method of clause 76, comprising: exiting the first control loop in response to a first exit condition being met.
Clause 78. The method of clause 77, wherein the first exit condition is met when a number of pauses initiated by the first control loop reaches a first pause count threshold.
Clause 79. The method of clause 76 or 77, wherein the first exit condition is met when the power threshold is at a maximum power threshold and the excess power condition occurs.
Clause 80. The method of any one of clauses 77-79, comprising: entering the second control loop in response to the exiting of the first control loop due to the first exit condition being met.
Clause 81. The method of clause 80, comprising: exiting the second control loop in response to a second exit condition being met; and reentering the first control loop in response to exiting of the second control loop in response to the second exit condition being met.
Clause 82. The method of any one of clauses 76-81, wherein the excess power condition occurs when the actual power of the motor assembly exceeds a power threshold, wherein, while executing the first control loop, the power threshold is increased during a pause.
Clause 83. The method of any one of clauses 76-82, comprising: exiting the first and/or second control loop upon completion of the firing stroke.
Clause 84. The method of any one of clauses 76-83, comprising: driving the firing assembly a predetermined distance through the firing stroke following a pause initiated in the first control loop such that a subsequent pause is not initiated at least until the firing assembly has traveled the predetermined distance.
Clause 85. The method of any one of clauses 76-84, comprising: driving the firing assembly a predetermined distance through the firing stroke following a pause initiated in the second control loop such that a subsequent pause is not initiated at least until the firing assembly has traveled the predetermined distance.
Clause 86. The method of any one of clauses 76-85, wherein the motor drive signal comprises a pulse width modulated (PWM) signal.
Clause 87. The method of clause 86, comprising: detecting the excess power condition in which a duty cycle of the PWM signal is greater than a predetermined duty cycle percentage value.

Having shown and described exemplary embodiments of the subject matter contained herein, further adaptations of the methods and systems described herein may be accomplished by appropriate modifications without departing from the scope of the claims. For instance, software methods can be realized in various types of hardware; and software methods can include additional steps; the surgical stapler 10 can be modified to include alternative and/or additional compatible features of other surgical staplers known in the art or yet to be developed. In addition, where methods and steps described above indicate certain events occurring in certain order, it is intended that certain steps do not have to be performed in the order described but, in any order, as long as the steps allow the embodiments to function for their intended purposes. Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

What is claimed is:

1. A surgical stapler comprising:
a firing assembly configured to translate along a longitudinal axis such that translation of the firing assembly in a distal direction is configured to deploy staples from an end effector;
a motor assembly mechanically coupled to the firing assembly and configured to drive the firing assembly along the longitudinal axis; and
a speed control circuit configured to:
output a motor drive signal to the motor assembly such that the motor drive signal is configured to drive the firing assembly to a target speed during a firing stroke,
detect, during the firing stroke, a speed error in which an actual speed of the firing assembly is less than a speed threshold that is less than the target speed, set the target speed to zero for a pause time duration in response to detecting the speed error,
increase the target speed above zero after the pause time duration, and
drive the firing assembly to the increased target speed a predetermined distance through the firing stroke.

2. The surgical stapler of claim 1, wherein the motor drive signal comprises a pulse width modulated (PWM) signal.

3. The surgical stapler of claim 2, wherein the speed control circuit is configured to dynamically adjust a duty cycle of the PWM signal during the firing stroke to drive the firing assembly to the target speed.

4. The surgical stapler of claim 2, wherein the speed control circuit is configured to:
detect, during the firing stroke, an excess power condition in which the duty cycle of the PWM signal is greater than a duty cycle threshold, and
set the target speed to zero for the pause time duration in response to detecting the excess power condition.

5. The surgical stapler of claim 2, wherein the speed control circuit is configured to dynamically adjust the duty cycle of the PWM signal based at least on an instantaneous speed error, a rate of change speed error, or a cumulative speed error.

6. The surgical stapler of claim 2, wherein the speed control circuit is configured to dynamically adjust the duty cycle of the PWM signal based at least on an instantaneous speed error measured as an instantaneous difference between the actual speed and the target speed, the instantaneous speed error being greater than a difference between the target speed and the speed threshold.

7. The surgical stapler of claim 1, wherein the speed control circuit is further configured to:
count a number of pause time durations during the firing stroke, and
drive the firing assembly to the increased target speed through completion of the firing stroke when the number of pause time durations is above a pause count threshold.

8. The surgical stapler of claim 1, wherein the speed control circuit is configured to:
set the target speed to an initial speed such that the firing assembly traverses an initial distance of the firing stroke driven to the initial speed.

9. The surgical stapler of claim 8, wherein the speed control circuit is configured to:
set the target speed after the pause time duration such that the target speed is set less than the initial speed and greater than zero.

10. The surgical stapler of claim 8, wherein the initial speed is approximately 12 mm/s to approximately 16 mm/s.

11. The surgical stapler of claim 8, wherein the initial speed is approximately 12 mm/s.

12. The surgical stapler of claim 8, wherein the motor drive signal is a PWM signal having less than 100% duty cycle in an unloaded firing condition as the firing assembly travels at the initial speed.

13. The surgical stapler of claim 1, wherein the pause time duration is approximately one second.

14. The surgical stapler of claim 1, wherein the speed control circuit is configured to:
detect, during the firing stroke, the speed error when the actual speed of the firing assembly is less than the target speed by the speed threshold and electrical current driving the motor assembly is greater than a current threshold.

15. The surgical stapler of claim 1, wherein the speed control circuit is configured to:
dynamically adjust power to the motor assembly to drive the firing assembly to the target speed during the firing stroke;
execute a first control loop during the firing stroke in which the speed control circuit is configured to monitor an actual power of the motor assembly and set the target speed to zero for the pause time duration each time an excess power condition occurs; and
execute a second control loop during the firing stroke in which the speed control circuit is configured to monitor the actual speed of the firing assembly and set the target speed to zero for the pause time duration each time the speed error is detected.

16. A surgical stapler comprising:
a firing assembly configured to translate along a longitudinal axis such that translation of the firing assembly in a distal direction is configured to deploy staples from an end effector;
a motor assembly mechanically coupled to the firing assembly and configured to drive the firing assembly along the longitudinal axis; and
a speed control circuit configured to:
output a motor drive signal to the motor assembly such that the motor drive signal is configured to dynamically adjust power to the motor assembly to drive the firing assembly to a target speed during a firing stroke,
execute a first control loop during the firing stroke in which the speed control circuit is configured to initiate a pause during the firing stroke in response to a detection of an excess power condition in which an actual power to the motor assembly is greater than a power threshold, and
execute a second control loop during the firing stroke in which the speed control circuit is configured to initiate a pause during the firing stroke in response to a detection of a speed error condition in which an actual speed of the firing assembly is less than a speed threshold that is less than the target speed, wherein the second control loop is distinct from the first control loop.

17. The surgical stapler of claim 16, wherein the speed control circuit is configured to:
exit the first control loop in response to a number of pauses initiated by the first control loop reaching a first pause count threshold.

18. The surgical stapler of claim 16, wherein the speed control circuit is configured to:
exit the first control loop in response to the power threshold being at a maximum power threshold and the excess power condition occurs.

19. The surgical stapler of claim 16, wherein the speed control circuit is configured to:
enter the second control loop in response to the exiting of the first control loop due to a first exit condition being met.

20. The surgical stapler of claim 16, wherein the speed control circuit is configured to:
exit the second control loop in response to a second exit condition being met; and
reenter the first control loop in response to exiting of the second control loop in response to the second exit condition being met.
